(19) United States
(12) Patent Application Publication
(10) Pub. No.: US 2018/0311390 A1
(43) Pub. Date: Nov. 1, 2018

(54) DEVICE FOR REDUCING ODOR IN ATHLETIC APPAREL STORAGE

(71) Applicant: DeoTech LLC, Schaumburg, IL (US)

(72) Inventors: Anthony Tamras, Schaumburg, IL (US); Kosta Plevritis, Schaumburg, IL (US); Drake Roberts, Schaumburg, IL (US); Anthony Portera, Palatine, IL (US)

(73) Assignee: DeoTech LLC, Schaumburg, IL (US)

(21) Appl. No.: 15/964,849

(22) Filed: Apr. 27, 2018

Related U.S. Application Data
(60) Provisional application No. 62/491,747, filed on Apr. 28, 2017.

(51) Int. Cl. A61L 9/014; A61L 9/12; A61L 9/04

(52) U.S. Cl. CPC A61L 9/014; A61L 9/12; A61L 2209/22; A61L 2209/14; A61L 9/044

(57) ABSTRACT

A device which reduces odor in athletic apparel storage, notably athletic or “gym” bags, lockers, and similar places where athletic apparel might be stored before it can be washed is disclosed. Chemical means both to reduce the odor of unwashed apparel by chemically neutralizing odor-causing compounds with a de-odorizer and to mask the odor of unwashed apparel with a masking scent, together or in the alternative, are disclosed. An exterior configuration for the device making it simple to use and appropriate for use in rugged conditions is also disclosed, as is an improved embodiment making the mode of the device (active or inactive) readily apparent.

### PRIORITY CLAIM

This application claims priority from U.S. Provisional Patent Application 62/491,747, “DEVICE FOR REDUCING ODOR IN ATHLETIC APPAREL STORAGE,” and the content of that application is hereby incorporated by reference.

This invention relates to a device which neutralizes odor-causing compounds in athletic apparel storage, notably athletic or “gym” bags, lockers, closets and similar places where athletic apparel might be stored before it can be washed. The device uses chemical means both to reduce the odor of unwashed apparel by chemically neutralizing odor-causing compounds and to mask the odor of unwashed apparel with a preferable fragrance. The device is configured to allow it to be used in rough conditions and to be activated and deactivated by simple means.

### BACKGROUND OF THE INVENTION

Human beings have been participating in athletic events for thousands of years. For just as long, the problem of saturating garments with perspiration and the resultant odor has presented itself. (The Ancient Greeks simply participated in athletic events with no clothes at all, which is not practical in modern society.)

In modern times, the problem is compounded by the fact that many people perform exercise or other athletic activity at gyms, fitness centers, community parks, or other places where they must travel to the athletic activity in street clothes, change to athletic apparel, perform the activity, and change back into street clothes, and then transport the used athletic apparel in a gym bag, duffle, or other container before it can be washed. The result is that the apparel remains saturated with perspiration and cannot dry out, thereby increasing the production of perspiration-related odor. A device which could ameliorate the development of such odor will be a useful invention.

Further, athletic apparel and other equipment worn on the body is frequently packed in such containers with small weights, skates either wheeled or bladed, sticks of various kinds for playing various games, and so forth. This produces a very rough environment where any device for ameliorating the development of such odor will be subject to potential impacts, cuts, or other rough treatment. A device for ameliorating the development of such odor which can stand up to such rough treatment will be a useful invention.

Finally, people in athletic environments are often in a rush and do not have time or inclination to perform complex or subtle operations when storing their equipment. A device which will ameliorate the development of such odor in a simple, easy and effective way will be a useful invention.

The present invention addresses these and other related concerns.

### SUMMARY OF THE INVENTION

Among the many objectives of the present invention is the provision of a device which can ameliorate the development of perspiration-related odor in bags and other containers where athletic equipment is stored.

Another objective of the present invention is the provision of a device which can ameliorate the development of perspiration-related odor in athletic environments, where rough handling is a possibility, without being easily damaged.

Yet another objective of the present invention is the provision of a device which can ameliorate the development of perspiration-related odor and can be easily and visibly activated, deactivated, and refilled.

These and other objectives of the invention (which other objectives become clear by consideration of the specification and drawings as a whole) are met by providing the device for reducing odor in athletic apparel storage set forth in the specification hereof.

### BRIEF DESCRIPTION OF THE DRAWINGS

FIG. 1(a) depicts a perspective view of the device in an activated configuration.

FIG. 1(b) depicts a perspective view of the device in a deactivated configuration.

FIG. 2 depicts a first side view of the device in the activated configuration

FIG. 3 depicts a second side view of the device in the activated configuration

FIG. 4 depicts an overhead view of the interior of the top half of the device.

FIG. 5 depicts an overhead view of the interior of the bottom half of the device.

FIG. 6 depicts a perspective view of the bottom half of the device.

### DESCRIPTION OF THE PREFERRED EMBODIMENTS

Reference will now be made in detail to several embodiments of the invention that are illustrated in accompanying drawings. Whenever possible, the same or similar reference numerals are used in the drawings and the description to refer to the same or like parts or steps. The drawings are in simplified form and are not to precise scale. For purposes of convenience and clarity only, directional terms such as top, bottom, left, right, up, over, above, below, beneath, rear, and front, may be used with respect to the drawings. These and similar directional terms are not to be construed to limit the scope of the invention in any manner. The words attach, connect, couple, and similar terms with their inflectional morphemes do not necessarily denote direct or intermediate connections, but may also include connections through mediate elements or devices.

For purposes of this application, there are two methods for remediating the odors associated with sweat, dirt, et cetera accumulated by athletic wear and equipment during exercise. The first method is to introduce a chemical which will chemically neutralize the odor-causing compounds, either by chemically reacting with them in such a way as to produce compounds which are not (as) odiferous, or by chemically bonding with them to prevent their dispersement and circulation through the air. Chemical neutralization of the actual odor-causing compounds by either method will be referred to herein as de-odorizing with a de-odorizer. The second method is to introduce a chemical with a pleasant scent which will mask, cover up, or otherwise distract from and/or overpower the scent of the odor-causing compounds. This will be referred to herein as masking odors with a masking scent. Any given substance used for purposes of the invention may be and/or contain a de-odorizer, a masking scent, or both.

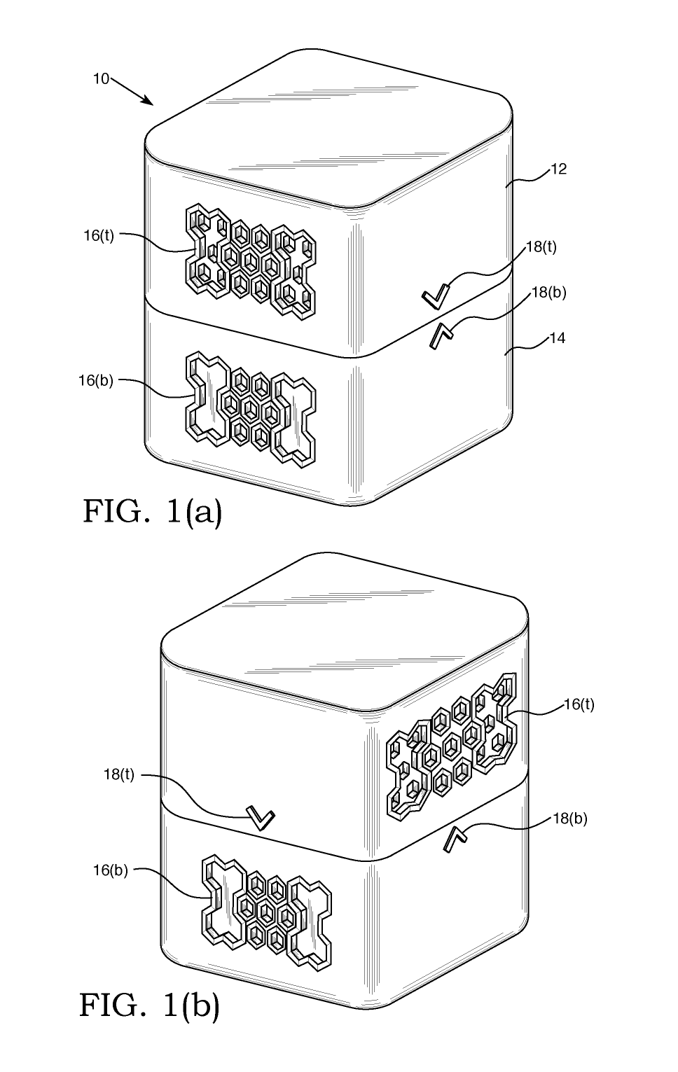

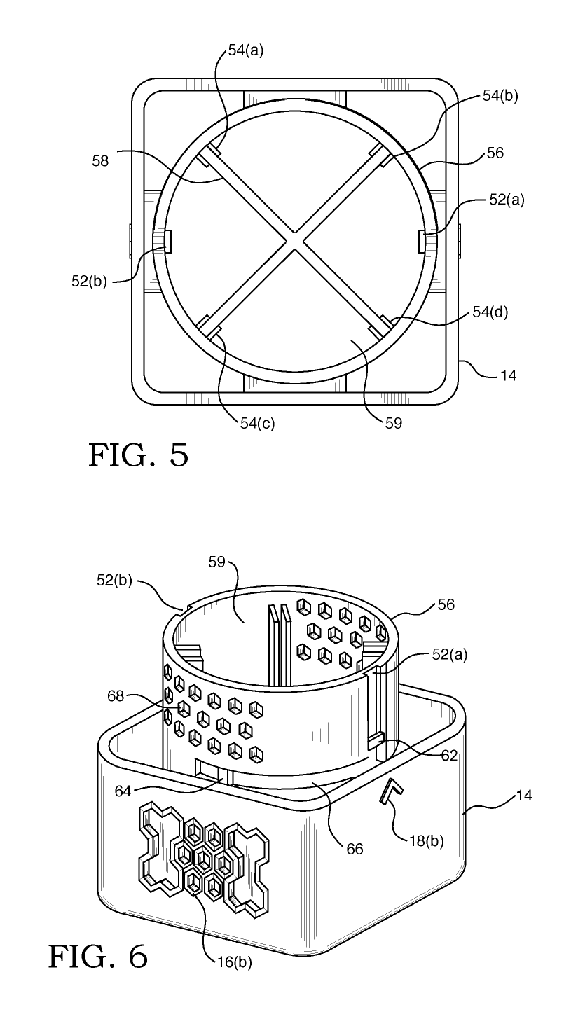

Now adding FIG. 1(a) and FIG. 1(b) to the consideration the configuration of the invention may be clearly understood. In FIG. 1(a), the device 10 is in an activated configuration. The faces of the device 10 which include vents 16(t) and 16(b) are aligned, indicating that the device 10 is activated (see FIG. 6 for corresponding interior vents.) Alignment indicators 18(t) and 18(b) are also aligned, indicating that the device 10 can be opened to replace or refresh the odor reduction means (See FIG. 5.)

In FIG. 1(b), the device 10 is in a deactivated configuration. The faces of the device 10 which include vents 16(t) and 16(b) are opposed, indicating that the device 10 is deactivated (see FIG. 6 for corresponding interior vents.) Alignment indicators 18(t) and 18(b) are also opposed, indicating that the device 10 cannot be opened to replace or refresh the odor reduction means (See FIG. 5.)

In both FIG. 1(a) and FIG. 1(b), the face of top 12 which is not tangent to a face of bottom 14 and the face of bottom 14 which is not tangent to a face of top 12, which may be considered the “top” and “bottom” of the device respectively, are featureless. It is slightly preferred, but not required, that these faces remain featureless and contain no operative elements, as this allows either or both of them to be marked with a logo, instructions, or any other desired content without fear of impairing the operation of the device 10 or obscuring the content with such features or operative elements.

It is preferred, but not required, that the edges and corners of the device (both top 12 and bottom 14) be slightly rounded so as to avoid scratching, ripping, or otherwise damaging the athletic apparel or equipment in the bag or other container, or the bag or other container itself. This also makes it less likely that the device 10 will catch on things and be damaged itself. It is strongly preferred, but not required, that the device 10 be cubical, as this provides surfaces which are easy to grip, easy to distinguish, and provide a simple way to positively identify and confirm the current configuration.

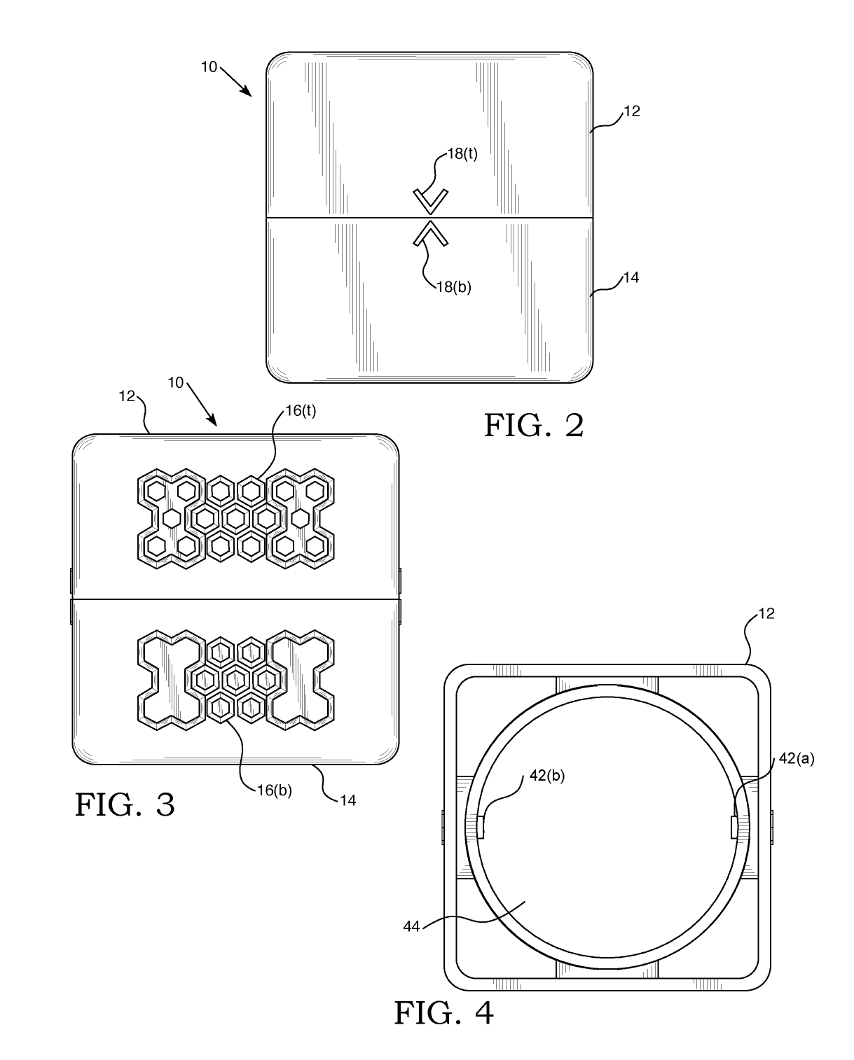

In FIG. 2, the sides of the device 10 which do not contain any vents are shown in an activated configuration. Alignment indicators 18(t) and 18(b) are aligned, indicating both that the device 10 is in the activated configuration and that it is in the proper configuration for opening. (See FIGS. 4 and 5.) It is strongly preferred, but not required, to include alignment indicators 18(t) and 18(b) as this allows for positive confirmation of the precise alignment necessary to open the device 10. It is strongly preferred that if alignment indicators 18(t) and 18(b) or some equivalent thereof are not included, that vents 16(t) and 16(b) are included only on faces which correspond in a single configuration, so as to provide a quick and easy means to ascertain which configuration the device 10 is in. Alternate methods may also be used, such as corresponding colors or patterns on the faces which show when the device 10 is in a particular configuration. Such indication is strongly preferred, but not required, as the user can always simply try to open the device 10 by using trial and error.

In FIG. 3, the sides of the device 10 which contain vents are shown in an activated configuration. Vents 16(t) and 16(b) are aligned, and the holes in vent 16(t) are communicating with cavity 59. (Not shown: see FIGS. 5 and 6 for more disclosure regarding vents 16(t) and 16(b).) Air may flow through the holes in vent 16(t), allowing the device 10 to perform its function of odor reduction (See FIG. 5 for a discussion of the means of odor reduction.) FIG. 4 shows the interior of top 12. Upper cavity 44 is a circular void whose interior diameter is slightly larger than the exterior diameter of vertical column 56. (Not shown: see FIG. 5.) The interior circumference of upper cavity 44 is interrupted by engagement members 42(a) and 42(b). It is preferred, but not required, that the engagement members 42(a) and 42(b) be diametrically opposed on the interior circumference of upper cavity 44, and that there be two of them. (See FIG. 5 for additional disclosure regarding the preferred configuration of engagement members 42(a) and 42(b), and the corresponding vertical and horizontal channels.) (See FIGS. 5 and 6.) It is required that all engagement members, whatever their number, be of the proper dimension to slide through the corresponding vertical and horizontal channels without more than minimal frictional resistance, and to be able to pass vertical and horizontal bosses 62 and 64 (See FIG. 6) with the expenditure of a reasonable amount of effort by a user of the device 10.

In FIG. 5, bottom 14 includes cavity 59, which is defined by the body of bottom 14 and its interior extension vertical column 56. Vertical column 56 is intended to be inserted into upper cavity 44 (Not shown: see FIG. 4.) The exterior circumference of vertical column 56 is interrupted by vertical channels 52(a) and 52(b). The interior circumference of vertical column 56 is interrupted by interior channels 54(a-d). Interior channels 54(a-d) are placed at ninety degree intervals around the interior circumference of vertical column 56, such that any given interior channel 54(a-d) has an interior channel diametrically opposed to it. Odor absorber 58 comprises a piece of absorbent material shaped into an “X” and sized such that its intersecting members are slightly smaller than the interior diameter of vertical column 56. Odor absorber 58 can be inserted into interior channels 54(a-d) and will be prevented from moving in the plane of the diameter of vertical column 56. When top 12 (not shown: see FIGS. 1a, 1b) is in place, odor absorber 58 cannot move in the dimension orthogonal to the diameter of vertical column 56 as it will be stopped by either the body of top 12 or the body of bottom 14.

It is preferred, but not required, that odor absorber 58 be sized such that it is only slightly shorter than the interior axis of vertical column 56. This will minimize the amount of movement possible in the axial direction and minimize vibration and impact wear and tear on odor absorber 58. Likewise, it is preferred that odor absorber 58 have intersecting members which are only slightly smaller than the diameter of vertical column 56 to minimize the amount of movement possible in the diametric plane and minimize vibration and impact wear and tear on odor absorber 58. It is required that odor absorber 58 have intersecting members which are longer than the distance from the interior end of opposing interior channels 54(a-d) so that odor absorber 58 will securely engage and be held in place by the interior channels 54(a-d).

Odor absorber 58 is comprised of an absorbent material which can absorb some quantity of a a de-odorizer, a masking scent, or both. There can be more than one substance, or the substance to be infused into odor absorber 58 can be a compound mixture to achieve both of these goals. When the device 10 is in an activated configuration, and vent 16(t) can communicate with cavity 59, air carrying diffuse perspiration can enter into the device 10. By diffusion, this air will expose the diffuse perspiration to odor absorber 58 and the de-odorizer infused into it. This will allow the de-odorizer infused into odor absorber 58 to chemically neutralize the compounds in the air which produce unpleasant odors.

Again by diffusion, this air will recirculate out of the device 10, carrying some of the masking scent if used, and/or the de-odorizer.

It is strongly preferred, but not required, that at least one of the substances infused into odor absorber 58 be an essential oil, as these are long-lasting, powerful in small quantities, and may serve as both a de-odorizer and a masking scent. Essential oils are also safer and more marketable than more complex synthetic compounds, although any appropriate substance or substances may be used.

It is preferred, but not required, that the vertical channels 52(a) and 52(b) be diametrically opposed on the exterior circumference of vertical column 56, and that there be two of them. It is required that all vertical channels, whatever their number, be of the proper dimension to allow the corresponding engagement members 42(a) and 42(b) to slide through them without more than minimal frictional resistance. It is required that there be a vertical channel for every engagement member on top 12 and that it be in the appropriate position to engage its corresponding engagement member.

In the preferred embodiment, there are at least two engagement members 42(a) and 42(b) which correspond to two vertical channels 52(a) and 52(b) and two horizontal channels 66 (see FIG. 6) as this allows for co-linear points of engagement. It is strongly preferred, but not required, for there to be at least two engagement members 42(a) and 42(b) and corresponding vertical and horizontal channels 52(a), 52(b), and 66. In an alternative embodiment, the device 10 will function as intended with only one engagement member et cetera, but it will be less secure and less resistant to damage or jamming (e.g. vertical column 56 may cant inside top 12, causing it to be difficult to separate the parts of the device.) A less secure embodiment may also be constructed using magnets, hook-and-loop fasteners, a pure friction fit, or any other reasonable means of ensuring top 12 and bottom 14 stay in the proper alignment, but a mechanical engagement is strongly preferred. This both improves durability and decreases the likelihood that the device 10 will open when it is not intended to open.

In FIG. 6, bottom 14 can be seen, absent top 12, to incorporate vertical column 56, which extends upward from the body of bottom 14 and defines cavity 59. Vertical channel 52(a) extends from the top of vertical column 56 to the base, partially interrupted by vertical boss 62. Horizontal channel 66 extends from the bottom of vertical channel 52(a) to a position 90 degrees around the circumference of vertical column 56, partially interrupted by horizontal boss 64. To assemble and use the device 10, odor absorber 58 is saturated with the appropriate substances, and then inserted in interior channels 54(a-d). Top 12 is then placed in contact with bottom 14 such that engagement members 42(a) and 42(b) engage corresponding vertical channels 52(a) and 52(b). Top 12 can then slide down vertical column 56, guided by the engagement members 42(a) and 42(b) and vertical channels 52(a) and 52(b). When the engagement members 42(a) and 42(b) pass the vertical bosses (e.g. vertical boss 62,) the two pieces of the device 10 will be locked together as the engagement members 42(a) and 42(b) will not pass back over the vertical bosses 62 without the expenditure of reasonable force.

To deactivate the device 10 and even more securely lock the pieces together, top 12 can be rotated ninety degrees (in the preferred embodiment, in a clockwise direction as viewed axially from above top 12.) This will cause the engagement members 42(a) and 42(b) to slide through horizontal channels 66 until they pass over the horizontal bosses (e.g. horizontal boss 64.) As with the vertical bosses 62, once this is done, the device 10 will remain in this configuration as the engagement members 42(a) and 42(b) will not pass back over the horizontal bosses 64 without the expenditure of reasonable force.

Interior vent 68 is placed on vertical column 56 such that when the device 10 is in an activated configuration, cavity 59 can communicate with vent 16(t), and thus with the outside atmosphere. When the device 10 is in a deactivated configuration, interior vent 68 will have rotated ninety degrees from vent 16(t) and cavity 59 will have no communication with the outside atmosphere.

In the preferred embodiment, when alignment indicators 18(t) and 18(b) are aligned, the device 10 can be opened by pulling top 12 away from bottom 14, as vertical boss 62 will not stop engagement members 42(a) and 42(b) from sliding up vertical channels 52(a) and 52(b) if sufficient force is applied. This is slightly preferred in that in this configuration the device 10 is securely closed when in the deactivated configuration, providing a secure “base state” where it is both deactivated (the vents are sealed) and locked together. However, it is acceptable to configure the device 10, in ways which will be obvious after this disclosure such as simply rotating any appropriate set of features during manufacture, in such a way that the device 10 is locked together when activated and can be opened when deactivated.

It is strongly preferred, but not required, that the holes in vent 16(b) be “blind” holes, which do not communicate with cavity 59, because in the preferred embodiment as described in FIG. 1(b) the rotation of top 12 and bottom 14 would not block such communication. If it is desired to have the holes in vent 16(b) communicate with cavity 59 and for there to be a true “deactivated” mode, it is required that some means of causing such communication to be interrupted when the device 10 is in the deactivated configuration be included. Alternatively, in an alternate embodiment where the holes in vent 16(b) communicate with cavity 59, but no such means is included, the “deactivated” configuration would be a “lower rate of activity” configuration in that the holes in vent 16(t) would not communicate with cavity 59 and thus the rate of air exchange would be lower but would not stop.

This application—taken as a whole with the abstract, specification, claims, and drawings—provides sufficient information for a person having ordinary skill in the art to practice the invention disclosed and claimed herein. Any measures necessary to practice this invention are well within the skill of a person having ordinary skill in this art after that person has made a careful study of this disclosure. It should be noted that the order of the steps and initial configurations in all disclosed embodiments may be varied as will be obvious to a person of ordinary skill in the art.

Because of this disclosure and solely because of this disclosure, modification of this device for reducing odor in athletic apparel storage can become clear to a person having ordinary skill in this particular art. Such modifications are clearly covered by this disclosure. Claims are not required in Provisional Patent Applications: the following claims are included to indicate the nature of the disclosed invention and should not be read as limiting the nature and scope of the claims of any utility application which may claim priority from this Provisional Patent Application.

1. A device for reducing odor in athletic apparel storage comprising:
a) A first piece having a first vent;
b) A second piece having a second vent, such that the second piece can be operably attached to the first piece, the first piece operably attached to the second piece together comprising a body having an interior space and an exterior space, the first vent and the second vent allowing the interior space and the exterior space to communicate; and,
c) An odor absorber contained within the interior space.

2. A device for reducing odor in athletic apparel storage as in claim 1 wherein the device has:
a) An activated position wherein when the device is in the activated position, the first vent and the second vent are in an aligned position and allow the interior space to communicate with the exterior space; and,
b) A deactivated position, wherein when the device is in the deactivated position, the first vent and the second vent are in an unaligned position and do not allow the interior space to communicate with the exterior space.

3. A device for reducing odor in athletic apparel storage as in claim 2 further comprising:
a) A vertical channel and a horizontal channel, located on the second piece, the vertical channel intersecting the horizontal channel such that they form a ninety-degree angle; and,
b) An engagement member located on the first piece, such that when the first piece is operably attached to the second piece, the engagement member can travel along the vertical channel until it reaches the horizontal channel.

4. A device for reducing odor in athletic apparel storage as in claim 1, wherein the odor absorber is an absorbent material saturated with a de-odorizer.

5. A device for reducing odor in athletic apparel storage as in claim 2, wherein the odor absorber is an absorbent material saturated with a de-odorizer.

6. A device for reducing odor in athletic apparel storage as in claim 3, wherein the odor absorber is an absorbent material saturated with a de-odorizer.

7. A device for reducing odor in athletic apparel storage as in claim 1, wherein the odor absorber is an absorbent material saturated with a masking scent.

8. A device for reducing odor in athletic apparel storage as in claim 2, wherein the odor absorber is an absorbent material saturated with a masking scent.

9. A device for reducing odor in athletic apparel storage as in claim 3 wherein the odor absorber is an absorbent material saturated with a masking scent.

10. A device for reducing odor in athletic apparel storage as in claim 1, wherein the odor absorber is an absorbent material saturated with an essential oil.

11. A device for reducing odor in athletic apparel storage as in claim 2, wherein the odor absorber is an absorbent material saturated with an essential oil.

12. A device for reducing odor in athletic apparel storage as in claim 3, wherein the odor absorber is an absorbent material saturated with an essential oil.
